(19) United States
(12) Patent Application Publication
(10) Pub. No.: US 2026/0297650 A1
(43) Pub. Date: Oct. 1, 2026

(54) Probe Synthesis Methods and Uses Thereof

(71) Applicant: McMaster University, Hamilton (CA)

(72) Inventors: Andrew McArthur, Hamilton (CA); Gerard Wright, Zurich (CA); Hendrik Poiner, Lynden (CA); Dirk Hackenberger, Hamilton (CA)

(21) Appl. No.: 19/576,429

(22) Filed: Mar. 24, 2026

Related U.S. Application Data
(60) Provisional application No. 63/779,884, filed on Mar. 28, 2025.

(51) Int. Cl. C12Q 1/6806; C12Q 1/6816

(52) U.S. Cl. CPC C12Q 1/6806; C12Q 1/6816

(57) ABSTRACT

This disclosure relates to synthesizing RNA probes for targeted enrichment of specific target nucleic acid sequences and methods of making and uses thereof. Resulting RNA probes may be used for hybridization-based sequencing methods, including but not limited to, next-generation sequencing or hybridization capture sequencing.

### FIELD OF INVENTION

The present disclosure relates to methods of synthesizing RNA probes for hybridization-based enrichment of target nucleic acid sequences and uses thereof. In particular, the disclosure relates to cost-effective in-house synthesis of labelled RNA probe sets from a synthetic oligonucleotide pool, in which the probe sequences may be selected to hybridize to any desired target(s), including, but not limited to, antimicrobial resistance genes (ARGs), viral sequences, and mobile genetic elements (MGEs).

### BACKGROUND

Shotgun DNA sequencing can detect at least most targets in a sample given sufficient depth. Groups have used this method to characterize antimicrobial resistance genes (ARGs) in environmental [1,2] and worldwide wastewater [3,4] resistomes. However, a limitation of this technique may be that target sequences in a mixed sample could represent several orders of magnitude less than 1% of the total DNA. For example, a single 1 kb target that makes up 1×10⁻⁶% of the DNA in a sample would require on the order of 10 Gbp of sequence data to obtain about 10-fold coverage of the target. Accordingly, deep sequencing without enrichment can be cost-prohibitive and data-intensive when targets are rare, because most reads will derive from non-target DNA and must be stored and computationally processed [5].

Accordingly, targeted enrichment using custom-designed nucleic acid probes may provide a technical and economic advantage over reliance on commercially available probe sets or untargeted sequencing approaches. In certain implementations, in-house probe design permits optimization of probe length, tiling density, thermodynamic parameters, redundancy filtering, and restriction-site architecture in a manner coordinated with downstream amplification and transcription workflows. This integrated design approach may reduce off-target hybridization, minimize probe-probe complementarity, and enable probe sets to be scaled to large target collections while remaining compatible with pooled oligonucleotide synthesis and in vitro transcription. In some cases, designing and manufacturing probes internally further permits rapid iteration of target content, control over probe-count limits for manufacturability, and implementation of algorithmic filtering criteria not available in fixed commercial panels. Such flexibility can improve enrichment efficiency, reduce reagent costs, and enable deployment in applications where commercially synthesized bait libraries would otherwise be cost-prohibitive, insufficiently customizable, or take too long to deliver.

Thus, it would be desirable to develop a method having one or more of the advantages outlined above.

The background herein is included solely to explain the context of the disclosure. This is not to be taken as an admission that any of the material referred to was published, known, or part of the common general knowledge as of the priority date.

### SUMMARY OF THE INVENTION

A targeted hybridization-based enrichment method has now been developed that allows for the robust detection of a broad range of specific, low-abundance nucleic acid targets with less sequencing.

In the present protocol, DNA from a sequencing library may be denatured, allowing biotinylated RNA probes complementary to target nucleic acid sequences within the library to hybridize. In one embodiment, streptavidin-coated magnetic beads capture these biotinylated RNA probes and their complementary DNA partners from the background (FIG. 1). This process increases the proportion of the target nucleic acid in a library, allowing one to sequence less yet detect more. Probe-capture protocols are useful for detecting thousands of targets in complex samples, including but not limited to targets such as antimicrobial resistance genes (ARGs), viral sequences, and sequences associated with mobile genetic elements (MGEs). Recognized herein are challenges associated with probe-capture strategies, including the cost of commercially supplied probes and the need to rapidly generate new probe sets as target sets evolve.

Thus, provided herein are methods, compositions, kits, and systems that enable cost-effective in-house synthesis of probe sets for hybridization-based targeted enrichment.

An aspect of the disclosure provided herein includes methods for designing probes for hybridization-based targeted enrichment of target nucleic acid sequences. In some embodiments, probe sequences are designed using a probe-design pipeline comprising tiling candidate probes across input target sequences, filtering candidate probes based on one or more sequence or thermodynamic constraints, filtering candidates based on off-target similarity, and iteratively reducing redundancy to obtain a final probe set.

Thus, in one aspect, a method of providing a single-stranded oligonucleotide probe set comprising a plurality of probes for hybridization-based targeted enrichment of target nucleic acid, the method comprising:
a) selecting a target nucleic acid;
b) generating candidate probe sequences to the target nucleic acid by tiling candidate probes across at least a portion of the target nucleic acid;
c) filtering the candidate probe sequences based on one or more sequence or thermodynamic constraints;
d) filtering the candidate probe sequences based on sequence similarity to one or more off-target nucleic acid sequence datasets;
e) reducing redundancy among remaining candidate probe sequences to obtain the probe set; and
f) optionally, synthesizing one or more of the probes from the probe set.

Another aspect of the disclosure provided herein includes methods of generating RNA baits for targeted enrichment of target nucleic acid sequences.

In accordance with this aspect, a method of generating labelled single-stranded RNA baits for hybridization-based targeted enrichment of target nucleic acid, the method comprising:
a) providing a plurality of probes each configured to hybridize to at least a portion of the target nucleic acid, appending an RNA polymerase promoter at the 5′ end of each probe and an amplification primer to the 3′ end of each probe to yield a pool of single-stranded DNA oligonucleotides;
b) amplifying the pool of single-stranded DNA oligonucleotides using (i) a first primer complementary to at least a portion of the RNA polymerase promoter and (ii) a second primer complementary to at least a portion of the amplification primer to generate double-stranded DNA templates; and
c) performing in vitro transcription of the double-stranded DNA templates in the presence of an RNA polymerase that recognizes the RNA polymerase promoter and mixture of four nucleotide triphosphates comprising adenine, guanine, cytosine and uracil bases, wherein at least one of the four nucleotide triphosphates is labelled, to generate labelled single-stranded RNA baits which are immobilizable.

Another aspect provided herein includes kits. The kits may comprise a variety of components. In some cases, the kits may comprise a collection (e.g., a pooled collection) of single-stranded oligonucleotides that include probe sequences configured to hybridize to a selected set of target nucleic acid sequences. In some cases, the kits may comprise one or more primers, a DNA polymerase, a restriction endonuclease, an RNA polymerase (e.g., T7 RNA polymerase), and/or a reagent for incorporating biotin into RNA probes (e.g., a biotinylated nucleotide). In some embodiments, the kits further comprise one or more nucleases (e.g., DNase I) and/or one or more purification reagents

Thus, a kit for generating biotinylated single-stranded RNA baits for hybridization-based targeted enrichment of target nucleic acid is provided. The kit comprises a pool of single-stranded DNA oligonucleotides, wherein each oligonucleotide comprises a probe sequence having an RNA polymerase promoter appended to the 5′ end of the probe and an amplification primer appended to the 3′ end of the probe. The kit may additionally comprise one or more of:
a) a first primer complementary to at least a portion of the RNA polymerase promoter;
b) a second primer complementary to at least a portion of the amplification primer;
c) a DNA polymerase suitable for PCR;
d) an RNA polymerase that recognizes the RNA polymerase promoter;
e) mixture of four nucleotide triphosphates comprising adenine, guanine, cytosine and uracil bases, wherein at least one of the four nucleotide triphosphates is biotinylated;
f) a restriction endonuclease; and/or
g) a DNase.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### DESCRIPTION OF DRAWINGS

Certain embodiments of the disclosure will now be described in greater detail with reference to the attached drawings in which:

FIG. 1 illustrates a targeted enrichment workflow in accordance with some embodiments of the present methods. Magnetic streptavidin beads may be used to bind biotinylated RNA baits, which may be hybridized to a complementary DNA partner. The beads may be pelleted and washed before amplification and sequencing. In some cases, this process may reduce the prevalence of non-complementary DNA before amplification and sequencing.

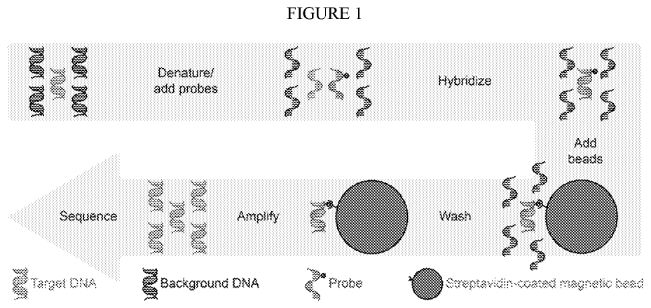

FIG. 2 illustrates a probe design strategy in accordance with some embodiments of the present methods. A) The workflow starts with 80-nt probes tiled every 4 nt across all sequences in the input, generated by a probe tiling tool (e.g., BaitsTools [8]). In this example, there are two similar input sequences (ARG 1 and ARG 2) and one that is unique (ARG 3), with no close homologs. B) During the basic filter, probes are filtered out if they do not meet specific physical characteristics (e.g., length of 80 nt, melting temperature >50° C., no ambiguous bases, etc.). Probes are also deduplicated, as redundant sequences in the design are not helpful during enrichment. C) Next, the probes are removed if they have off-target hits in a background database (e.g., the BLASTN nt database). D) Finally, redundant probes are iteratively collapsed at different levels of sequence identity (e.g., 79, 78, 77, 76 . . . ) to single representatives until the total number of probes is below a predefined cut-off. Note that ARGs 1 and 2 have lower probe density than ARG 3. However, due to ARGs 1 and 2's similarity, each probe designed against one will also hybridize with the other, resulting in a final probe density similar to that of ARG 3.

FIG. 3 illustrates the probe set statistics for representative allCARD and clinicalCARD probe sets synthesized according to the methods described herein. a) Number of probes remaining after each filter employed during design. b) Physical statistics of probes in each set.

FIG. 4 illustrates a representative in silico probe analysis in which probe sequences from example allCARD and clinicalCARD probe sets were aligned using BLAST against (a) genes in the CARD database and (b) a subset of those genes identified as clinically relevant.

FIG. 5 illustrates certain representative 12.5% Urea-PAGE gels comparing size distributions of commercial probe sets and in-house synthesized probe sets generated according to the methods described herein, demonstrating an expected smeared size distribution consistent with biotinylated RNA baits for (A) an ARG-targeting probe set, (B) a virus-targeting probe set, and (C) an MGE-targeting probe set.

FIG. 6 illustrates representative enrichment efficiency comparisons between example treatments in certain embodiments. Rarefaction analysis of detected gene counts at subsampling depths of 100 k reads up to 3 M reads is shown for (a) wastewater samples and (b) soil samples. Shaded areas represent the 95% confidence interval. (c) Average enrichment factor±1 standard deviation for example probe sets across sample types. In this illustrative embodiment, enrichment factor is defined as the number of reads mapped to a reference gene database after enrichment with a given probe set divided by the number of reads mapped to the reference gene database in corresponding shotgun sequencing data.

FIG. 7 illustrates representative analyses of the top 20 antimicrobial resistance (AMR) genes ranked by overall read count and their associated read proportions across sample types, including (a) wastewater samples and (b) soil samples, in certain example embodiments. For clarity, long gene names are shown using corresponding short identifiers from the Comprehensive Antibiotic Resistance Database (CARD).

FIG. 8 illustrates representative analyses of A) overlap and B) gene coverage distribution in wastewater samples following enrichment using commercial and in-house synthesized probe sets in certain example embodiments. In this illustrative implementation, the analyzed genes correspond to those included in a specific reference database version used during probe set design.

FIG. 9 illustrates representative analyses of A) overlap and B) gene coverage distribution in soil samples following enrichment using commercial and in-house synthesized probe sets in certain example embodiments. In this illustrative implementation, the analyzed genes correspond to those included in a specific reference database version used during probe set design.

FIG. 10 illustrates representative rarefaction curve extrapolations showing detected gene counts at a sequencing depth of 10 million reads in certain example embodiments. Shaded areas represent the 95% confidence interval.

FIG. 11 illustrates representative correlations between enriched read counts and corresponding shotgun sequencing read counts for example gene targets in certain embodiments, including associated R² values. (a) In a wastewater dataset, specified gene targets meeting defined inclusion criteria were analyzed for two sampling periods. (b) In a soil dataset, one probe set was not analyzed in certain samples because the applied inclusion criteria resulted in insufficient mapped reads. Specific gene counts and sample conditions shown are illustrative of particular experimental datasets.

FIG. 12 shows the prevalence data for plasmids and chromosomes for ARGs in CARD in exemplary embodiments of the disclosure. ARGs are shaded according to their annotated AMR mechanism in CARD.

FIG. 13 shows a probe synthesis workflow as used in some embodiments. The first step may be to add amplification primers to either end of every unique probe sequence in the set. This allows PCR amplification of the synthesized oligo pool before LguI restriction endonuclease treatment and subsequent T7 RNA transcription in the presence of biotinylated UTP.

FIG. 14 shows the workflow for library preparation, indexing, and enrichment treatments as used in some embodiments. Each set was sequenced on its own sequencing run.

FIG. 15 shows the distribution of the number of similarities between read and reference, based on CIGAR strings from the BAM file output by the CARD RGI bwt module as used in some embodiments. The internal line of each violin plot indicates the median of the distribution.

FIG. 16 shows the comparison of enrichment efficiency of clinically relevant ARGs between allCARD and clinicalCARD probe sets as used in some embodiments. (a) Overlap analysis showing ARGs detected by allCARD and clinicalCARD, for both with at least 100 reads at different subsampling depths. (b) Coverage analysis showing the distribution of coverage for ARGs detected by both probe sets with at least one read at a depth of 3 M. In the November sample, there were 226 such ARGs. In the March sample, there were 176.

### DETAILED DESCRIPTION

### I. Definitions

Unless otherwise indicated, the definitions and embodiments described in this and other sections are intended to be applicable to all embodiments and aspects of the present disclosure herein described for which they are suitable, as would be understood by a person skilled in the art. It is also to be understood that the terminology used herein is for the purpose of describing particular aspects only and is not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings, such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of′ or “one or more” of the listed items is used or present. The word “or” is intended to include “and” unless the context clearly indicates otherwise.

The abbreviation “e.g.” is derived from the Latin exempli gratia and is used herein to indicate a non-limiting example. Thus, the abbreviation “e.g.” is synonymous with the term “for example.”

The term “enrichment”, as used herein, refers to increasing the amount of a target nucleic acid (e.g. DNA) and increasing the ratio of target nucleic acid relative to non-target nucleic acid in a sample.

The term “probe”, as used herein, refers to a single-stranded oligonucleotide which can specifically hybridize to a target nucleic acid, either in solution or as a surface-bound nucleic acid. In some embodiments, probes are single-stranded RNA molecules that may be labelled, e.g. biotinylated; such probes may also be referred to herein as “RNA baits”.

The term “tail” or “flanking tail”, as used herein, refers to non-target nucleotides flanking a probe or bait sequence that are not intended to hybridize to a target nucleic acid sequence, such as primer, promoter or any adapter sequences appended to a probe to facilitate amplification or processing.

The term “hybridization” refers to the specific binding of a nucleic acid to a complementary nucleic acid via Watson-Crick base pairing.

The term “target nucleic acid”, “target nucleic acid sequence” or “target sequence”, as used herein, refers to a nucleic acid sequence to be enriched, captured, detected, and/or analyzed from a sample or sequencing library. Target nucleic acids comprise DNA and/or RNA and may be derived from any organism or genetic element, including bacterial, archaeal, viral, and eukaryotic sources, as well as sequences associated with MGEs. Such target nucleic acid sequences are selected based on the sample type and the target sequences that may be of interest within the given sample, the presence of which is desired to be determined with the sample.

The term “mobile genetic element” or “MGE”, as used herein, refers to genetic elements that are capable of moving within a genome and/or between genomes, and include, for example, plasmids, transposons, integrative and conjugative elements (ICEs), integrons, transposons, insertion sequences, genomic islands, prophage and phage-related elements, and fragments or combinations thereof. The term “mobilizable mobile genetic element” or “mobilizable MGE”, as used herein, refers to an MGE that is capable of horizontal transfer between cells, either autonomously or with assistance from one or more helper elements, and includes, for example, conjugative plasmids, mobilizable plasmids, integrative and conjugative elements (ICEs), integrative and mobilizable elements (IMEs), transposons, integrons, and fragments or combinations thereof.

### II. Overview

Provided herein are methods, kits and systems related to targeted enrichment of nucleic acid targets from metagenomic, environmental, clinical, or other complex nucleic acid mixtures. The methods comprise the use of oligonucleotide probes designed to hybridize to selected target nucleic acid sequences. The target nucleic acid sequences may comprise, for example, antimicrobial resistance genes (ARGs), viral sequences, and/or sequences associated with mobile genetic elements (MGEs), among many other possible targets. The oligonucleotide probes may comprise one or more features, including but not limited to one or more RNA polymerase promoter sequences, primer sequences, or a combination thereof. In some cases, the oligonucleotide probes may be amplified. In some cases, one or more features of the oligonucleotide probes may be modified. In some cases, the oligonucleotide probes may be amplified to form DNA templates for use to initiate RNA transcription of the probe sequences. In some cases, the RNA transcripts may be produced during transcription. In some cases, the RNA transcripts may comprise at least a portion of a sequence corresponding to one or more target nucleic acid sequences. The kits described herein may comprise various components used in the methods described herein.

An aspect of the disclosure provided herein includes methods of synthesizing RNA baits for targeted enrichment of target nucleic acid sequences. In some cases, the methods may comprise selecting a single-stranded oligonucleotide from a collection of single-stranded oligonucleotides designed to bind to one or more target nucleic acid sequences. In some cases, the methods may comprise adding (appending) or providing an RNA polymerase promoter to one end of the single-stranded oligonucleotide (such as the 5′ end) and appending an amplification primer to the opposite end (e.g. the 3′ end) of the single-stranded oligonucleotide. The RNA polymerase promoter and/or amplification primer may be added directly to the end of the single-stranded oligonucleotide. The term “directly” refers to the addition of the promoter and/or primer to the oligonucleotide with no intervening sequence therebetween.

In some embodiments, the single-stranded oligonucleotide originates from a collection of custom-designed single-stranded synthetic oligonucleotides representing an RNA bait set configured to hybridize to a selected set of target nucleic acid sequences. In some embodiments, the promoter sequence provided to one end of the oligonucleotide may be any suitable promoter, including a T7 promoter sequence. In some embodiments, the promoter sequence includes up to three additional guanine bases directly downstream of the transcription initiation site to improve transcription efficiency and uniformity (more than three guanine bases may be included, however, there is no advantage to doing so). In some embodiments, the amplification primer sequence is designed for subsequent amplification of the oligonucleotide. In some embodiments, the amplification primer sequence contains a restriction enzyme cut site proximal to the probe sequence. The restriction enzyme cut site is not particularly limited. In some embodiments, the restriction enzyme cut site is LguI, such that the oligonucleotides can be cleaved to remove flanking sequence after amplification and before RNA transcription. In some embodiments, the oligonucleotide probes are >40 nucleotides in length. In some embodiments, the RNA transcription is carried out using T7 RNA polymerase. In some embodiments, the DNA digestion step includes the use of one or more nucleases to digest and remove residual DNA template. In some embodiments, the resulting RNA transcripts or baits are purified, for example using one or more purification steps. In some embodiments, the RNA baits can be captured using streptavidin-coated magnetic beads in a subsequent hybridization step. In some embodiments, the RNA baits are used in a targeted enrichment protocol for the detection of target nucleic acid sequences in environmental or clinical samples. In some embodiments, the RNA baits are used in targeted sequencing or gene panel assays to selectively enrich target sequences from a metagenomic sample. In some embodiments, the RNA baits are used for hybridization-based sequencing methods, including but not limited to next-generation sequencing (NGS) or hybridization capture sequencing.

In some cases, the methods may comprise amplifying the single-stranded oligonucleotide using primers complementary to the promoter sequence and the amplification primer sequence to generate double-stranded DNA templates. In some cases, the methods may comprise cleaving at least a portion of the amplification primer-sequence from the amplified templates using a restriction endonuclease, thereby preparing the templates for subsequent RNA transcription. In some cases, the methods may comprise performing an RNA transcription reaction on the templates using an RNA polymerase, wherein labelled, e.g. biotinylated or otherwise suitably labelled, nucleotides are incorporated into the synthesized RNA transcript. In some cases, the methods may comprise performing a DNA digestion step to remove any residual DNA from the transcription product. In some cases, the resulting RNA transcript or bait corresponds to target nucleic acid sequences and can be used in targeted enrichment applications.

Another aspect provided herein includes kits. The kits may comprise a variety of components. In some cases, the kits may comprise a collection of single-stranded oligonucleotides designed to bind to a selected set of target nucleic acid sequences. In some cases, the kits may comprise a restriction enzyme. In some cases, the kits may comprise an RNA polymerase such as T7 RNA polymerase. In some cases, the kits may comprise a protein with DNA endonuclease activity.

It will be understood that any component defined herein as being included may be explicitly excluded by way of proviso or negative limitation, such as any specific compounds or method steps, whether implicitly or explicitly defined herein.

In certain aspects, the disclosure provides (i) methods for designing probe sequences and (ii) methods for generating labelled, e.g. biotinylated, single-stranded RNA probes from oligonucleotide pools for hybridization-based targeted enrichment. The probe generation methods described herein are not limited to probes designed using the methods and may be practiced independently thereof.

### III. Probe Design

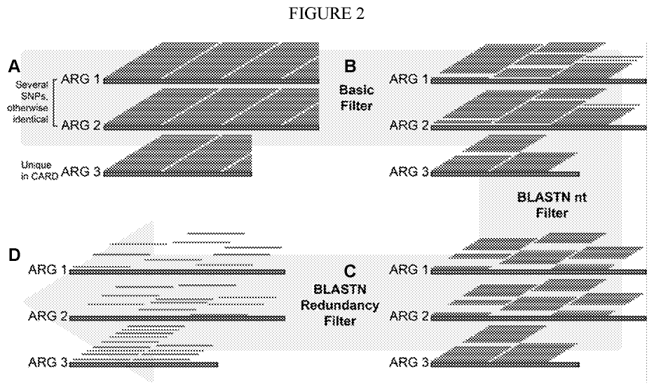

In some embodiments, probe sequences are generated by a pipeline that tiles candidate probes across target sequences that have been selected for detection in a given sample. The selection is based on sample type and the target sequences within the sample type which are desired to be detected. Candidate probes are filtered based on one or more sequence and thermodynamic constraints, filters candidates based on off-target similarity to one or more background databases and reduces redundancy until a final probe set is obtained. An exemplary workflow is illustrated in FIG. 2. The disclosure is not limited to the specific parameter values used in any one implementation.

Generating the candidate probe sequences comprises tiling probes of a selected probe length (e.g., greater than about 40 nucleotides such as about 40 to 200-400 nucleotides, or 40-120 nucleotides, or 60-100 nucleotides, or 70-90 nucleotides, such as about 80 nucleotides) across the target nucleic acid sequences at a defined step size (i.e., the distance between the start of one probe to the start of the next probe, e.g., 1-10 nucleotides), thereby producing an initial candidate set with redundancy prior to filtering. In some embodiments, and as one non-limiting implementation of the probe-design pipeline, candidate probes are generated as about 80 nucleotide probes tiled across input target nucleic acid sequences at a tiling distance of about 4 nucleotides.

In some embodiments, probes are randomly subsampled rather than tiled to form the candidate set with redundancy prior to filtering.

Filtering based on one or more sequence or thermodynamic constraints comprises excluding candidates that: (i) contain ambiguous bases, (ii) are predicted to have a melting temperature (Tm) below a threshold Tm determined, for example, via the nearest-neighbor model, and/or (iii) include one or more restriction endonuclease recognition sites used in a downstream probe synthesis workflow (e.g., a type IIS restriction endonuclease site such as an LguI site). In some embodiments, filtering may be based on predicted off-target similarity comprising aligning candidate probe sequences against one or more off-target nucleic acid sequence datasets (e.g., a microbial genome database, a host genome database, or a combination thereof) and excluding candidates having sequence similarity to one or more sequences in an off-target dataset that is above a threshold identity, for example, a threshold sequence identity of at least 80% or greater over a threshold length of at least 40-50 nucleotides of identity over the entire probe sequence. An “off-target nucleic acid sequence dataset” includes sequences which may be similar to the target sequence but are not the target sequence, and may further be present in the metagenome from which the target sequence is being isolated (e.g., human in wastewater). In some embodiments, reducing redundancy comprises iteratively collapsing clusters of probe candidates (e.g. candidates having 80% or greater sequence similarity that share decreasing identity with one another to a single representative candidate and/or collapsing a probe cluster that exhibit at least 80% complementarity over a probe length of at least 40 nucleotides to a single representative candidate until a target probe count is reached.

In certain implementations, the initial candidate set is deduplicated, candidate probe length is confirmed, candidates containing ambiguous bases are removed, and candidates having a perfect complement within the candidate set are removed.

In some embodiments, candidates are further filtered by thermodynamic and manufacturability constraints, including requiring a predicted melting temperature greater than about 50 degrees C. and excluding candidates containing a restriction endonuclease recognition site used in a downstream cleavage step, such as an LguI recognition site. These values are illustrative and non-limiting, and other probe lengths, tiling distances, melting temperature thresholds, and restriction-site exclusions may be used.

In some embodiments, off-target similarity filtering comprises comparing candidate probes against one or more background databases using BLASTN, for example, against the NCBI nt database. In certain implementations, probes are removed if they exhibit greater than about 80% identity in an alignment shorter than about 50 nucleotides to an off-target sequence, e.g. bacterial sequence. In certain implementations, probes are also removed if they exhibit greater than about 80% similarity across more than about 50 nucleotides to a viral, archaeal, or eukaryotic sequence, unless the candidate is a perfect match to the target nucleic acid sequence under comparison. These thresholds are provided as non-limiting examples of objective filtering criteria and do not limit the invention to any particular database, taxonomic grouping, aligned length, or percentage threshold.

In some embodiments, redundancy reduction is performed by BLASTN self-comparison of the remaining candidate probes followed by iterative collapse of clusters of probes with shared sequence identity to single representative sequences. In one non-limiting example, clusters of probes that share identity across about 79 nucleotides are collapsed to single representatives, with the process repeated at progressively lower identity levels until a desired probe count is reached or a manufacturability threshold for pooled synthesis is satisfied. In certain implementations, the final probe set is constrained to a probe-count cap of about 20,000 to about 40,000 probes to facilitate pooled oligonucleotide synthesis and downstream transcription at scale. The foregoing complementarity lengths and probe-count caps are illustrative only, and other similarity metrics, collapse orders, and target probe-count caps may be used.

In some embodiments, the probe-design pipeline provides a technical improvement over naive tiling and selection by generating probe sets that satisfy manufacturability constraints (e.g., a probe-count cap) while maintaining coverage of the target sequence space. For example, the filtering and redundancy-reduction operations reduce computational burden and probe-set size by eliminating redundant candidates and collapsing highly similar candidates to representative probes, thereby producing an output probe set suitable for pooled synthesis and in vitro transcription.

In some embodiments, the pipeline further improves hybridization capture performance by reducing predicted off-target hybridization and/or probe-probe interactions. For example, candidates may be screened against one or more background databases to exclude probes having off-target similarity above a threshold, and candidates may be screened for self-complementarity and/or complementarity to other probes, thereby reducing predicted non-productive hybridization. The resulting probe sets can improve capture efficiency and/or coverage uniformity in hybridization-based enrichment workflows relative to unfiltered, highly redundant tiling sets.

Following generation of a probe set, the probes may be synthesized using well-established synthesis techniques for use in a method of generating RNA baits in accordance with a further aspect of the present invention.

### IV. Synthesis of Labelled Single-Stranded RNA Baits

In some embodiments, the probes designed as described above are used in a method of generating labelled single-stranded RNA baits for hybridization-based targeted enrichment of target nucleic acid and are designed to hybridize to at least a portion of the target nucleic acid. An RNA polymerase promoter is appended to a 5′ end of each probe and an amplification primer is appended to the 3′ end of each probe to yield a pool of single-stranded DNA oligonucleotides.

In some embodiments, each synthetic oligonucleotide in the pool has a total length of greater than about 80 nucleotides, for example, 80-350 nucleotides, 100-150 nucleotides, 110-140 nucleotides, 115-130 nucleotides or about 120 nucleotides.

In some embodiments, appending of the polymerase promoter to the 5′ end and amplification primer at the 3′ end of each probe is conducted using established molecular recombination techniques. For example, restriction enzyme digestion followed by ligation may be used in which the enzyme creates complementary “sticky ends” on the promoter and probe which are joined by a DNA ligase. Other methods may also be employed including Gibson assembly and in-fusion cloning. In other embodiments, this is accomplished in silico, and the resulting plurality of sequences are ordered as an oligo pool.

In certain implementations, the RNA polymerase promoter appended to the 5′ end of the probe may be any suitable promoter such as a T7, T3 or SP6 promoter. In one embodiment, the promoter includes three or more additional guanine bases at or directly adjacent (downstream) to the transcription initiation site, and preferably 3 guanine bases, to reduce transcription efficiency variability.

In some embodiments, the amplification primer contains a restriction endonuclease recognition site positioned directly proximal to the probe sequence. Any restriction site may be incorporated; however, preferred sites include restriction sites for enzymes having an unbalanced cut that remove all primer sequence as well. Examples include but are not limited to LguI BspQI and SapI restriction sites. This permits cleavage of at least a portion of the flanking sequence following amplification and prior to transcription to generate RNA bait transcripts that substantially correspond to the probe sequence rather than the full flanked oligonucleotide. This architecture is illustrative and non-limiting; other promoters, added bases, restriction sites, cleavage positions, and overall oligonucleotide lengths may be used.

In some embodiments, the amplification primer is selected by an objective algorithmic workflow. In one non-limiting example, a plurality of candidate primers having terminal restriction sites, such as terminal LguI sites, are generated with primer lengths selected to yield a final oligonucleotide length of about 120 nucleotides when combined with the promoter and probe sequence. The candidate primer sequences are then compared, for example using BLASTN, against concatenated promoter-plus-probe sequences representing the oligo pool design, and the candidate primer having the fewest matches is selected to reduce predicted interactions with sequences in the pool. In certain implementations, the reverse complement of the selected primer sequence is appended to the probe sequence. These algorithmic steps are non-limiting examples, and other selection criteria, alignment tools, scoring methods, or oligonucleotide lengths may be used.

Once prepared, the pool of single-stranded DNA oligonucleotides is amplified via the polymerase chain reaction (PCR) using (i) a first primer complementary to at least a portion of the RNA polymerase promoter and (ii) a second primer complementary to at least a portion of the amplification primer to generate double-stranded DNA templates. Preferred conditions for conducting the PCR include about 12 cycles with denaturation at about 98 degrees C. for about 10 s, annealing at about 60 degrees C. for about 30 s, and extension at about 72 degrees C. for about 15 s. Following amplification, the DNA double-stranded templates may be treated with a restriction enzyme that targets the restriction site introduced via the amplification primer to remove at least a portion of any flanking sequence on the template prior to transcription to generate RNA bait transcripts that substantially correspond to the probe sequence rather than the full flanked oligonucleotide.

Transcription of the amplified double-stranded DNA templates is then performed in vitro under suitable conditions. Transcription is conducted in the presence of an RNA polymerase that recognizes the RNA polymerase promoter appended to the probe, and nucleotide triphosphates (e.g. adenine, cytosine, uracil and guanine), wherein at least one of the nucleotide triphosphates (e.g. uracil) is labelled with an affinity label to allow for immobilization thereof, for example, biotinylated, to generate labelled single-stranded RNA baits.

### V. Advantages and Technical Effects

In some embodiments, the methods described herein provide one or more of the following advantages:
a) Tail-free transcripts: Reducing or eliminating non-complementary flanking tails on the probes can improve hybridization kinetics by reducing unfavorable interactions at the termini.
b) Single-stranded RNA probes: Producing labelled, e.g. biotinylated, single-stranded RNA probes can increase effective probe availability and reduce probe-probe interactions during hybridization when compared to methods that prepare double-stranded, complementary probes.
c) Custom primer binding sequence: Selecting a non-universal amplification primer binding sequence with low predicted affinity to sequences within the oligo pool can improve amplification evenness and representation of probe sequences in the pool by avoiding nonspecific binding during oligo-pool amplification.

### EXAMPLES

The following non-limiting examples are illustrative of the present disclosure:

### Example 1: In-house synthesis of ARG bait set

A software package was written—the Comprehensive Antibiotic Resistance Probe Design Machine (CARPDM)—to generate a stringently filtered probe set with minimal off-target enrichment from the CARD v3.2.5 protein homolog model (allCARD) ARGs. This software package will run alongside all new releases of CARD, ensuring there is always an open-source and up-to-date probe set to enrich ARGs from any sample. Additionally, a curated list of 323 clinically relevant ARGs was created and a smaller probe set (clinicalCARD) was also generated with the same program, providing a more focused alternative to the allCARD probe set, as the full complement of ARGs may not be necessary for many projects in healthcare settings. To address the cost of commercial probes, a protocol that allows in-house synthesis of any probe set from a Twist Biosciences oligo-pool was developed. With this strategy, researchers can synthesize thousands of reactions worth of any probe set for a one-time fee lower than that of a typical 16-reaction kit from a commercial supplier.

### Materials and Methods

ARG selection for allCARD. Only ARGs curated as protein homolog models were included from CARD v3.2.5 (n=4,661), as these ARGs did not confer resistance via the acquisition of mutations (i.e., CARD's protein variant models). Including mutation-based ARGs in the design have enriched wild-type alleles, as enrichment probes readily hybridized over a single mutation, diluting the sequencing effort.

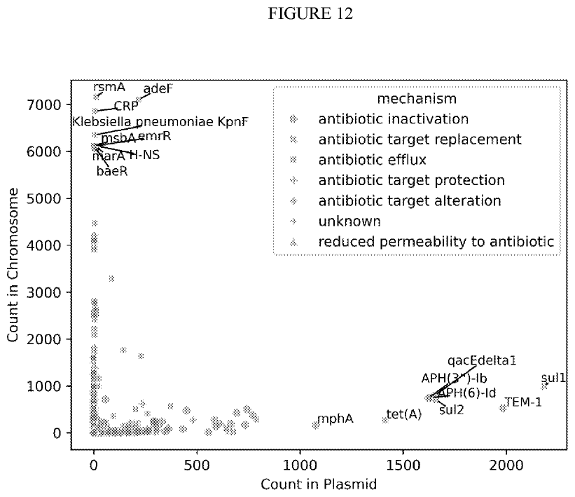

ARG selection for clinicalCARD. The CARD prevalence data v4.0.0 (10) aided in identifying clinically relevant ARGs. This data set was a collection of 221,175 sequencing assemblies from 377 pathogens with associated ARGs identified by CARD's Resistance Gene Identifier (RGI) software [10]. This data set included a collection of 21,079 completely sequenced chromosomes and 41,828 completely sequenced plasmids. Preliminary examination of this data illustrated that the distribution of ARGs was almost binary, i.e., each ARG predominantly occurred in either plasmids or chromosomes, but not both (FIG. 12). It also showed that ARGs with a higher occurrence of plasmids were far more likely to be clinically relevant [11-14]. As such, the first list of candidates clinically relevant ARGs included any ARG in CARD with at least 10 occurrences in plasmids, yielding 237 ARGs. Ten were chosen as a cutoff as it minimized the false positive inclusion of highly prevalent but clinically irrelevant ARGs, such as species-specific efflux mechanisms. Any ARG with >5% but <95% prevalence in any ESKAPE pathogen [15,16] (Enterococcus faecium, Staphylococcus aureus, Klebsiella pneumoniae, Acinetobacter baumannii, Pseudomonas aeruginosa, and Enterobacter species) was also added to this list, yielding a further 124 ARGs. Finally, 42 ARGs identified as clinically significant via a literature review were added, as they were included in publications investigating similar questions [17.18]. Overall, a wide net was cast, erring on the side of false positives and resulted in a draft set of 403 candidate ARGs. This set was manually curated based on CARD prevalence data, considering the species they appeared in, the genomic context (whether it was mobile, i.e., on a plasmid), the drug classes they impact, the environmental overlap of the host organism with humans, and the relative risk each host imposed as a pathogen. This approach was developed following recent ARG risk frameworks developed for metagenomic data [17,18]. In short, the manual curation selected for mobile ARGs that confer resistance to antibiotics used for therapy in humans was present in a human pathogen and was likely to colonize humans or animals. After curation, 323 ARGs were deemed clinically relevant and included in the clinicalCARD probe set design.

Probe design. CARPDM (Comprehensive Antibiotic Resistance Probe Design Machine) was written in Python, save for the first step, which employed the program BaitsTools [8] to tile probe sequences along ARG sequences. Probes designed by CARPDM had an 80 nt length as per the prior validated ARG bait set for CARD v1.0.1. This was also an ideal size for adding amplification primers while remaining below the 120 nt cut-off for the base level of a Twist oligo-pool if synthesizing probes in-house. The probes also had an extremely high density before filtering, with a tiling distance of 4 nt along ARG nucleotide sequences. This increased redundancy in the probe set and made it more robust against stringent downstream filtering. As input, the allCARD design used all 4,661 CARD v3.2.5 protein homolog model ARGs, while clinicalCARD's design used the curated 323 clinically relevant ARGs.

There were three filtering steps after the initial probe construction (FIG. 2). The first was a filter based on sequence, in which the probes were deduplicated, confirmed as 80 nt, contained no ambiguous bases, had no perfect complements within the set, had a Tm>50° C., and contained no LguI restriction endonuclease cut sites. The last condition is essential for the synthesis protocol. Since bacteria were likely to be the most abundant organism in most metagenomic samples [19], the second filter was a BLASTN search against the nt database to minimize off-target enrichment and remove probes with >80% identity over <50 nt to any bacterial sequence, as any probe similar to a bacterial sequence over >50 nt was likely the ARG itself. This filter also removed probes with >80% similarity over >50 nt to any viral, archaeal, or eukaryotic sequence unless they match perfectly (i.e., likely sequence contamination by an ARG).

The last filter removed redundancy in the probe set, as it used BLASTN to compare the probes against each other. To remove probes that could have bound to each other rather than the target DNA, any probes that were found to be complementary over the entire 80 nt were collapsed to a single representative. After removing complementary probes, CARPDM determined if the number of probes in the set were below a pre-defined cut-off. For allCARD, a cut-off of 40,000 probes was arbitrarily set, while for clinicalCARD, it was set to 20,000 probes. If not below the cut-off, CARPDM collapsed probes with 79 nt of complementarity to a single representative, repeating the process with decreasing length of complementarity until below the cut-off.

In silico analysis of probe sets. BLASTN was used to compare the probe sets to the ARG sequences. Using Python and NumPy [21], “target” arrays of zeros were constructed. Each of these arrays represented a single ARG against which the probe set was designed. After aligning all probes against the input set of target sequences with BLASTN, arrays of ones (matches) and zeros (mismatches) were constructed for each probe: target alignment. These arrays were then added to the corresponding target array at the start position of the alignment. Repeating this for every probe: target alignment yielded the tallied coverage of each nucleotide position by probes. This coverage array was used to compute summary statistics.

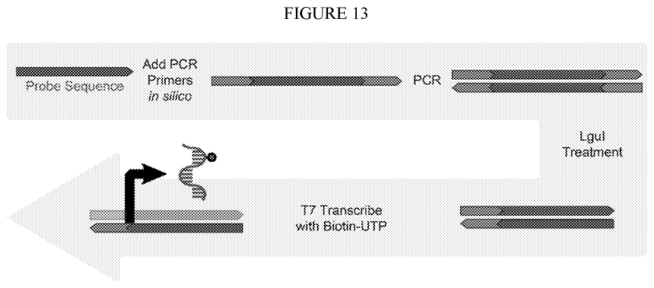

Probe synthesis. The starting material used for the in-house synthesis of probes was a custom oligo pool (Twist Biosciences, San Francisco, CA). The synthesis scale of the pool ordered varied based on the total number of unique probes in the pool. For allCARD, a synthesis scale that allowed 32,000-36,000 unique sequences was ordered, while for clinicalCARD, this scale was 16,000-20,000. Each unique oligo in this pool's design contained an individual probe sequence between two other sequences that were the same across all oligos, such that the final length is 120 nt. The first of these flanking sequences was a T7 transcription start site with three extra guanines to reduce transcription efficiency variability [22]. The second was a unique primer sequence chosen by the CARPDM program to minimize the probability of interactions with the probe sequences of the pool. This primer was required to contain an LguI cut site directly proximal to the unique probe sequence, such that it can be cleaved after amplification of the pool and before T7 transcription (FIG. 13).

The last part of CARPDM's pipeline was to convert the designed probe sequences that would typically be ordered as a probe set from a commercial supplier into oligo pool sequences that would be ordered from Twist Biosciences. To do this, CARPDM appended a T7 transcription start site with three extra guanines to one end of every probe sequence. It then created every possible primer with a terminal LguI cut site of the correct length to yield a final oligo of 120 nt when appended to the opposite side. It compared these putative primers to the concatenated T7/probe sequences using BLASTN and selected the primer with the fewest matches as the second amplification primer, appending the reverse complement to the opposite side of each probe sequence. The resulting sequences were ordered as an oligo-pool from Twist Biosciences (San Francisco, CA), and the amplification primers were ordered from Integrated DNA Technologies (Coralville, IA). For probe synthesis, sixteen 50 μL PCRs were conducted in parallel, each with 1 ng oligo pool input using 0.5 μL Phusion polymerase with HF buffer (Thermo Fisher Scientific, Waltham, MA), 1 μM of each primer, and 0.2 mM dNTPs (Thermo Fisher Scientific, Waltham, MA). Cycling conditions were initial denaturation at 98° C. for 30 s, 12 cycles of 98° C. for 10 s, 60° C. for 30 s, 72° C. for 15 s, and final extension at 72° C. for 10 m. Reactions were purified with the QIAQuick Nucleotide Removal Kit (Qiagen, Hilden, Germany), pooling eight reactions per column and eluting each in 30 μL. The elutions were then pooled, and their concentrations were quantified via Qubit 1×dsDNA HS assay (Thermo Fisher Scientific, Waltham, MA).

Four 50 μL restriction endonuclease treatments were then performed in parallel, each with 2 μg PCR input and 2 μL FastDigest LguI (Thermo Fisher Scientific, Waltham, MA). These reactions were then incubated at 37° C. for 2 hours, followed by heat inactivation at 65° C. for 5 min. Parallel reactions were then pooled over a single Qiagen MinElute PCR Purification column (Qiagen, Hilden, Germany), eluted in 10 μL, and quantified via Qubit 1×dsDNA HS assay.

Finally, T7 transcription reactions were performed with up to 1 μg of purified LguI digest product, although similar yields were achieved with as little as ~250 ng. For this, HiScribe T7 High Yield RNA Synthesis Kit (New England Biolabs, Ipswitch, MA) was used according to the manufacturer's instructions, with one-third of the UTP concentration comprised of Bio-16-UTP (Thermo Fisher Scientific, Waltham, MA). The 20 μL reactions were incubated for 16 h at 37° C., after which 68 μL RNase-free H2O was added with 10 μL DNase I Buffer and 2 μL DNase I (New England Biolabs, Ipswitch, MA). The resulting mix was incubated for 15 m at 37° C., after which the RNA probes were purified using the Monarch RNA Cleanup kit (50 μg; New England Biolabs, Ipswitch, MA). Finally, concentrations were quantified via Nanodrop (Thermo Fisher Scientific, Waltham, MA), and probes were diluted to 100 ng/μL. One hundred nanograms of each probe set were then analyzed on a 12.5% Urea-PAGE gel stained with SYBR-Gold (Thermo Fisher Scientific, Waltham, MA). A successful reaction was indicated by a smeared band of a slightly higher molecular weight than 80 nt compared to the DNA marker and no remaining band at 120 nt. The probes should have appeared at a slightly higher molecular weight than 80 nt due to the incorporation of biotin and the extra molecular weight contribution to RNA molecule weight from the 2′ hydroxyl group. The resulting smear indicated the successful incorporation of biotin into the probes depending on uracil content and the inherently stochastic nature of biotinylated vs non-biotinylated uracil insertion. A 120 nt band would indicate the remaining oligo pool if present, which should have been removed by the DNase treatment.

Samples. Two wastewater samples and three soil samples were selected to test the efficacy of the newly constructed probe sets with sequencing. The wastewater samples were 24 h aggregate influent samples from the city of Hamilton (Ontario, Canada) Wastewater Treatment Plant (7 Nov. 2022 and March 2023). DNA was extracted from 50 mL wastewater samples within 24 h of sampling using the DNeasy PowerWater Kit (Qiagen, Hilden, Germany) according to the manufacturer's protocols, each with an associated H2O control. Soil samples were collected from three different environments selected to represent distinct levels of human impact. The first was taken on 10 Jun. 2016, from Holman Island in the Northwestern Territories of Canada, a pristine environment with little human influence. The second was taken on 6 Mar. 2023, from a local wetland in an urban setting in Hamilton (Ontario, Canada), representing an environment with middling human impact. The third was taken on 27 Feb. 2023, from a high-traffic pedestrian area frequented by smokers outside of a Hamilton (Ontario, Canada) hospital, a setting with heavy human influence. All soil samples were stored at −80° C. until processing. DNA was extracted from 250 mg soil samples using the Qiagen DNeasy Powersoil Kit (Qiagen, Hilden, Germany) according to the manufacturer's protocols alongside an associated H₂O control.

Sample processing, enrichment, and DNA sequencing. Commercially synthesized probes (CARD v1.0.1 probe set only) and reagents for all enrichments were purchased from Daicel Arbor Biosciences (Ann Arbor, MI). In addition, probes for the allCARD, clinicalCARD, and CARD v1.01 sets were synthesized in-house as outlined above. While allCARD and clinicalCARD probe sets were only synthesized in-house, commercially synthesized CARD v.1.0.1 probes allowed direct comparison with the identical CARD v.1.0.1 probe set synthesized in-house.

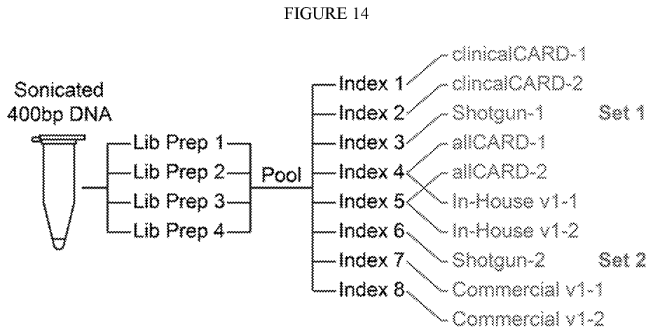

For each sample, extracted DNA was quantified via NanoDrop (Thermo Fisher Scientific, Waltham, MA), diluted, and sonicated to an average size of 400 bp using Covaris G-tubes (Woburn, MA). From this, libraries were prepared in quadruplicate from 1 μg DNA using the NEBNext Ultra II ligation kit (New England Biolabs, Ipswich, MA) according to the manufacturer's protocols. These libraries were then pooled before redistributing to half-size indexing reactions using NEBNext Multiplex Oligos for Illumina (New England Biolabs, Ipswitch, MA). Sample libraries were then enriched in two batches. The first batch performed allCARD and clinicalCARD enrichments, while the second batch performed commercial and in-house CARD v1.0.1 enrichments (FIG. 14). All enrichments were performed according to the manufacturer's protocols (version 5.02) using the Daicel Arbor myBaits v5 kit and reagents in a half-size reaction format with a 24 h hybridization at 62° C., maximum library input, 250 ng of probes, and 14 cycles of post-enrichment reamplification. In-house probes were diluted with RNase-free water to the same concentration as Daicel Arbor's (100 ng/μL) before use in the protocol. Each enriched library was compared to the same sample without enrichment.

Before sequencing, libraries were quantified in triplicate using NEB Luna Universal Probe qPCR Master Mix (New England Biolabs, Ipswitch, MA), Illumina PhiX standard (San Diego, California), and the following primers, all ordered from Integrated DNA Technologies (Coralville, IA):

P5:
AATGATACGGCGACCACCGA,
P7:
CAAGCAGAAGACGGCATACGA,
and
probe:
/56-FAM/CCCTACACG/ZEN/ACGCTCTTCCGATCT/3IABkFQ/.

Cycling conditions were initial denaturation at 95° C. for 3 m, followed by 40 cycles of denaturation at 95° C. for 15 s and annealing/extension at 60° C. for 1 m. The resulting concentrations were used to make individual pools for each enrichment set above, alongside one replicate of the shotgun (without enrichment) libraries (FIG. 14). Paired-end reads of 2× 150 bp were obtained from each pool using an Illumina NextSeq 2000 (San Diego, California), with samples having an average depth of 4.47 M clusters sequenced (minimum of 3.26 M clusters and a maximum of 6.71 M clusters).

Analysis and visualization. All analyses were performed with custom Python scripts and visualized with the ggplot2 package in R. For rarefaction analysis, libraries were subsampled every 100,000 paired-end reads up to 3 M using seqtk v1.3. Fastp v0.23.2 performed initial read trimming, quality control, and deduplication for these subsamples without merging. CARD's RGI bwt v6.0.2 tool with KMA v1.4.9 then mapped reads to reference sequences in CARD v3.2.5. ARGs were classified as present if they had more than 100 reads mapping (regardless of the breadth of coverage of the reference sequence). Regression lines were determined without extrapolation using the ggplot local estimated scatterplot smoothing function with geom_smooth. Since one cannot extrapolate a locally estimated function, a standard log-linear model was used when extrapolating the predicted number of ARGs detected at a sequencing depth of 10 M. GNU parallel v20161222 [25], the Python pandas v1.5.3 library [26], and BioPython v1.78 were used heavily for these analyses.

For read distribution analysis, custom Python scripts used the CARD RGI bwt output to count the number of reads that mapped to each ARG in soil and wastewater samples, respectively. The top 20 most prevalent ARGs (i.e., ARGs with the highest number of reads mapping) in each sample source (soil and wastewater) were kept for the figure, while all others were collapsed to the “other” category. This cut-off was chosen to keep figure legends readable while providing discriminatory power between the performance of different probe sets. Mean counts of the two replicates were used to determine the number plotted. The distribution of percent identity between read and reference for each sample was determined by a custom Python script that parsed the CIGAR strings in the BAM file that accompanies the RGI bwt output, where the percent identity was calculated as the number of nucleotide matches/151×100. Replicates were pooled for this analysis.

In a clinicalCARD vs allCARD overlap analysis, a custom Python script determined clinically relevant ARGs detected by allCARD or clinicalCARD with ≥100 mapped reads in both replicates at each subsampling depth. A similar approach was used to determine the overlap between the in-house vs commercially synthesized CARD v1.01 probe sets. However, to compare these sets, only ARGs included in the initial design (i.e., in CARD v1.0.1) were considered. Coverage analysis only considered clinically relevant ARGs detected by both clinicalCARD and allCARD with at least one read at a subsampled sequencing depth of 3 M paired-end reads in both replicates. Python scripts determined each ARG's coverage using the CARD RGI bwt output from the rarefaction analysis. Once again, for a similar analysis comparing the commercial and in-house synthesis, only ARGs from CARD v1.0.1 were considered.

For a wastewater read correlation analysis, only clinically relevant (i.e., in clinicalCARD) ARGs detected with at least one read in both shotgun and enriched in each sample were considered. All ARGs were included from soil samples, while clinicalCARD analysis was omitted due to the sparsity of detected ARGs. R² values were calculated using scikit-learn v1.2.2 [28]. The cmlA1 read number correction outlined below was accomplished by summing the number of reads attributed to all closely related cmlA variants (i.e., cmlA1, 4, 5, 6, 8, and 9) in each relevant treatment and manually adding the corresponding values to the plot.

### Results

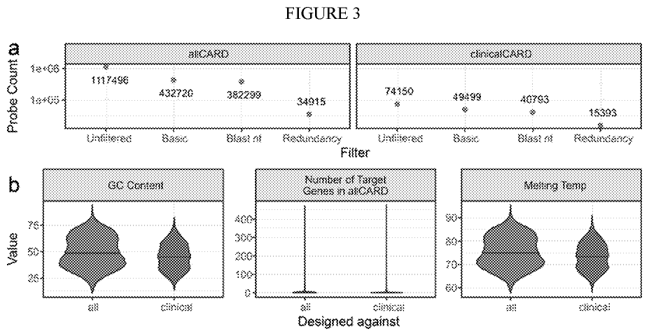

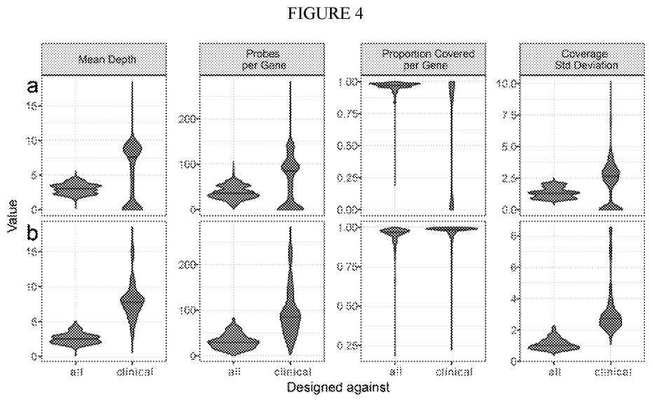

Probe design and synthesis. After design and filtering, allCARD contained 34,915 unique probe sequences covering 4,661 ARGs. Alternatively, clinicalCARD contained 15,393 unique probes, covering 323 ARGs (FIG. 3). No probe set had zero coverage of an ARG against which it was designed. When analyzing probes against all ARGs curated as CARD protein homolog models (FIG. 4a), the median value of all metrics other than the proportion covered per ARG was higher in the clinicalCARD probe set. The reason for clinicalCARD's bimodal distribution compared to allCARD is that it was only designed against the clinically relevant subset. Therefore, the clinically irrelevant genes (e.g., tolC and H-NS) had no probes aligning since they were not included in the initial design. However, despite only being designed against 323 ARGs, over 50% of CARD ARGs had coverage of >75% by clinicalCARD. When analyzing both probe sets against the clinically relevant ARGs in clinicalCARD (FIG. 4b), median values in clinicalCARD were higher than allCARD in every metric.

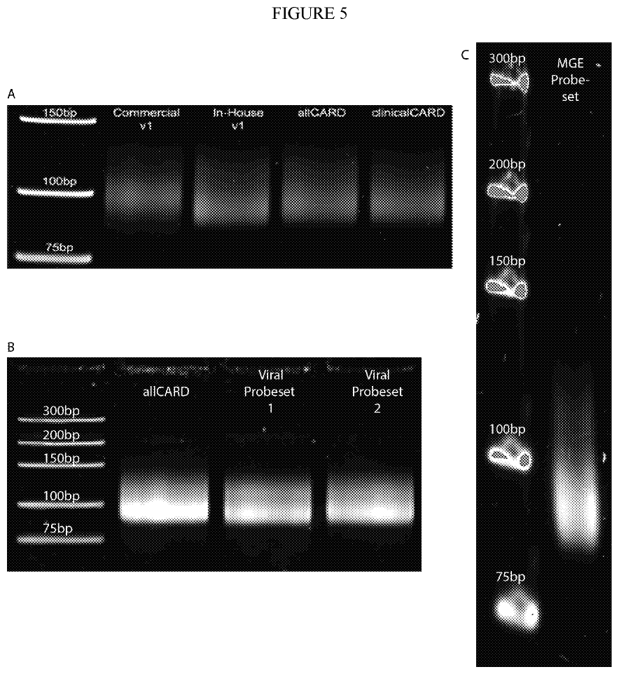

The increased in silico performance of clinicalCARD is a consequence of the smaller initial set of reference sequences. As such, it did not have to remove as many probes during the redundancy filter (FIG. 3a) and ended up with more redundancy and superior coverage of the ARGs against which it was designed. Probes in clinicalCARD had a lower median GC content and melting temperature. A higher proportion of probes also target only a single ARG in clinicalCARD relative to allCARD (FIG. 3b). After synthesis, a urea-PAGE gel shows a smear above 80 nt due to the stochastic incorporation of biotin into the probe (FIG. 5a). A comparison of all pools against the commercially synthesized version indicates a similar size distribution, with a slight bias toward lower molecular weights in the in-house synthesized probe set.

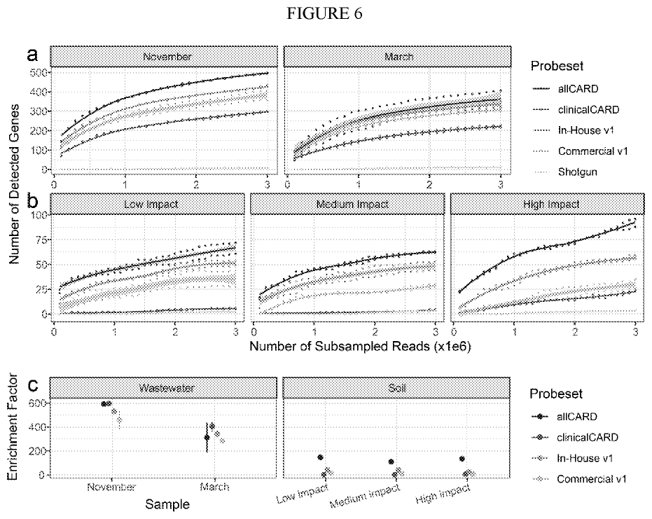

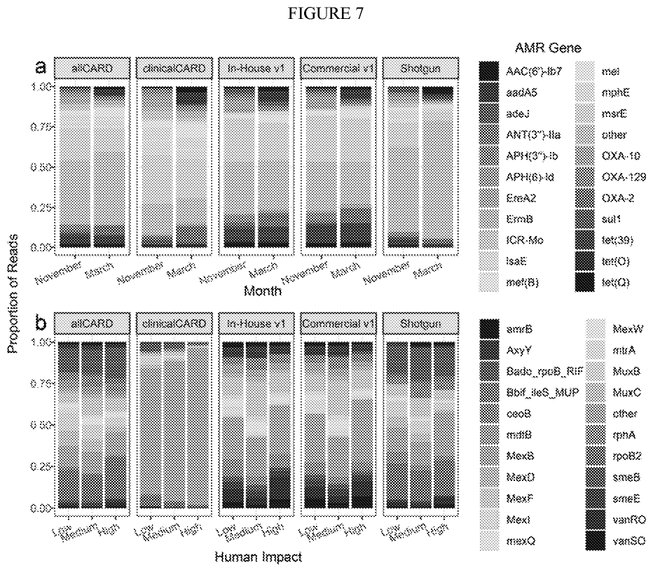

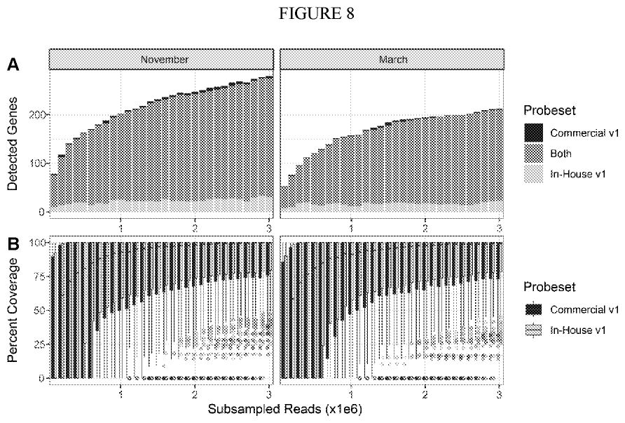

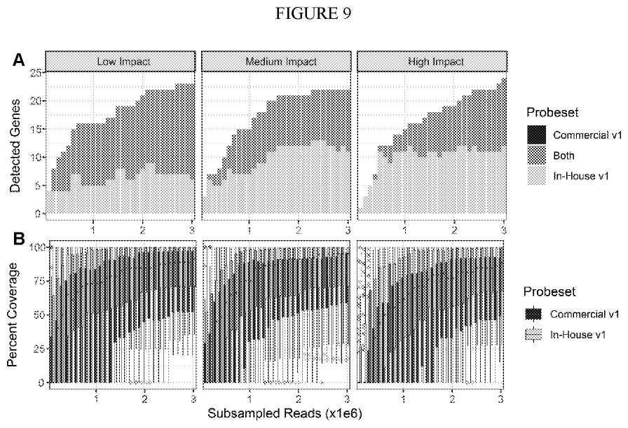

Probe testing. Five samples in five different treatments were employed to determine the efficacy of the probes when enriching for targets. No blanks had sufficient sequencing depth to be analyzed at even the lowest subsampling depth, indicating negligible contamination. First, the non-inferiority of the in-house synthesized probe set relative to the commercial option was established. After subsampling every 100 k paired-end reads up to 3 M with analysis by the CARD RGI bwt tool [29], the number of ARGs with >100 mapped reads was determined for each depth in each sample (FIG. 6). This analysis illustrated that the in-house synthesized probes detected more ARGs at the same sequencing depth in every sample. This effect was more pronounced in the soil samples, where the in-house synthesized probe set detected as many as double the number of ARGs detected by the commercially synthesized probe set, despite both having identical probe sequences based on CARD v1.01. Yet, the in-house synthesized probe set had an almost identical distribution of the top 20 detected ARGs after enrichment compared to the commercially synthesized set (FIG. 7), indicating consistent enrichment efficiency between ARGs in the commercial and in-house synthesized sets. These results were further complemented by analyzing the overlap between ARGs detected by each set at each subsampling depth and the associated coverage distribution for all detected ARGs (FIGS. 8 and 9). In these analyses, enrichment with the in-house synthesized probes detected ARGs with less sequencing effort than commercially synthesized probes and had greater coverage of detected ARGs.

Enrichment with all probe sets detected vastly more ARGs in wastewater samples than by sequencing without enrichment. allCARD detected the most, with up to 498 different ARGs, and clinicalCARD detected the least, with up to 300 ARGs. However, clinicalCARD efficiently retained the highest abundance of ARGs, evidenced by the similar distributions between allCARD and clinicalCARD in FIG. 7a. allCARD detected by far the greatest number of ARGs in soil samples, up to 96 in the high-impact hospital grounds. clinicalCARD did not detect a substantial number of ARGs in any soil sample, except for that taken from the high-impact hospital grounds, where it detected 24.

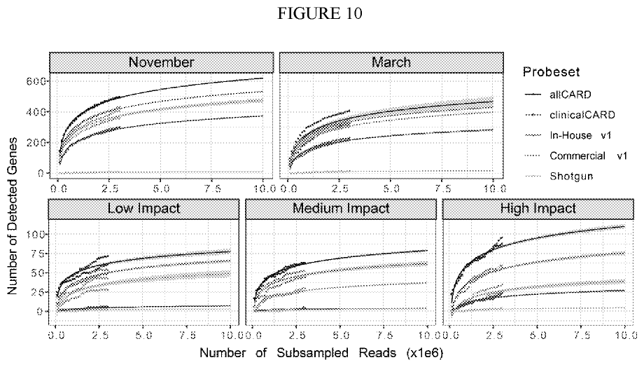

When subsampled to the same depth, the enrichment factor (i.e., the number of enriched reads mapped to CARD divided by the number of shotgun reads mapped to CARD) of different probe sets shows a more consistent value within each sample than expected, given the high performance of allCARD at detecting the most ARGs (FIG. 6c). This is especially evident in wastewater, where clinicalCARD is on par with, if not outperforming, allCARD in terms of enrichment factor despite detecting far fewer ARGs in the rarefaction curve. There was a 400-600-fold enrichment in the wastewater samples in the November sample, depending on the probe set, which decreased to 200-400-fold for the March sample. Soil samples had a markedly lower enrichment factor than wastewater, hovering between 0- and 200-fold enrichment depending on the probe set. Yet, enrichment was consistent between samples for different probe sets in soil. Based on the rarefaction curves (FIG. 6), ARG detection in most samples begins to plateau by a sequencing depth of 3 M paired-end reads. Extrapolation of these rarefaction curves to a sequencing depth of 10 M paired-end reads indicates that at a subsampling depth of 3 M, 70%-80% of the diversity of ARGs that may be detected at 10 M was captured (FIG. 10).

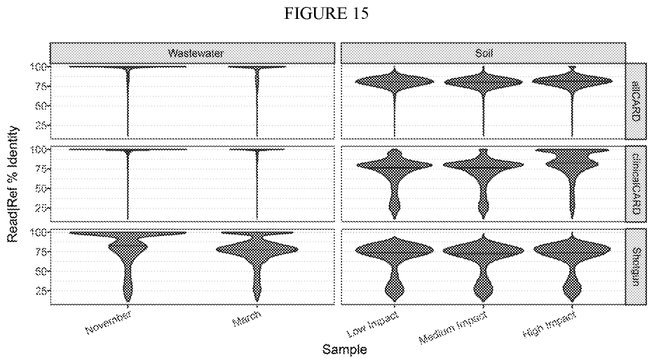

There were differences when comparing the top 20 most prevalent ARGs in soil and wastewater (FIG. 7). First, all top 20 ARGs in wastewater data, save for adeJ and tetQ, were in the set of clinically relevant ARGs. However, in soil samples, not a single top 20 ARG was included in the clinically relevant set, which explains the lack of enrichment for any top 20 ARGs in soil by the clinicalCARD probe set. Most dominant ARGs in the soil samples were found across various species and associated with efflux pumps (e.g., mex genes) or transcriptional regulators (e.g., mtrA and vanR/S). Moreover, the percent identity of the ARG-associated reads relative to the CARD reference sequences in the soil samples was lower than those from wastewater samples (FIG. 15). Before enrichment, the wastewater sample had a mixture of high-(>90%) and mid-identity (60%-90%) reads relative to their references in CARD, but the soil samples had exclusively mid-to low-identity (<60%) reads. After enrichment, wastewater samples had almost exclusively high-identity reads in both the clinicalCARD and allCARD enrichments. In soil samples, enrichment with allCARD was selected heavily for mid-identity reads, but clinicalCARD was preferentially selected for high-identity reads, especially in the sample from the high-impact hospital grounds.

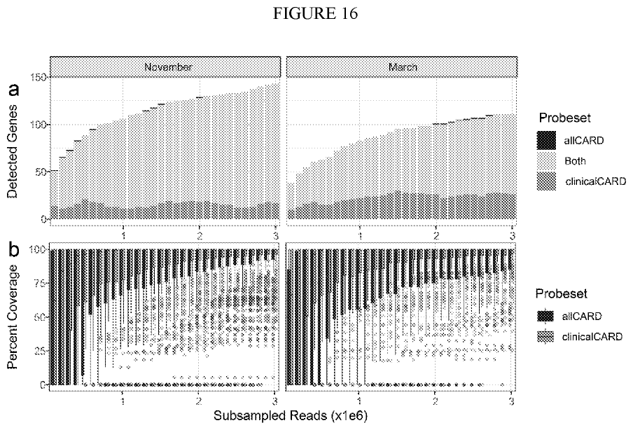

allCARD vs clinicalCARD. The wastewater samples were used to examine clinicalCARD's efficacy relative to allCARD at enriching the clinically relevant ARGs it was designed against, as clinically relevant ARGs had the highest abundance in the wastewater samples. Overall, clinicalCARD detected an average of 16% more ARGs than allCARD in the November wastewater sample and 35% more in the March sample at each subsampling depth, with very rarely an ARG detected uniquely by allCARD (FIG. 16a).

To assess the breadth of coverage of individual ARGs after enrichment with allCARD or clinicalCARD, clinically relevant ARGs detected by both probe sets in both replicates at a subsampling depth of 3 M paired-end reads were analyzed. The distribution of coverage of these ARGs at each subsampling depth for allCARD and clinicalCARD was plotted (FIG. 16b). clinicalCARD delivered 100% coverage of at least 50% of the ARGs detected in the November sample at a subsampling depth of 1 M reads, while allCARD took 1.5 M reads to do the same. In the March sample, clinicalCARD took only 700 k reads to reach this level of detection, while allCARD required 2.1 M reads. Based on this analysis, clinicalCARD delivers better coverage of more clinically relevant ARGs at a lower sequencing depth than allCARD.

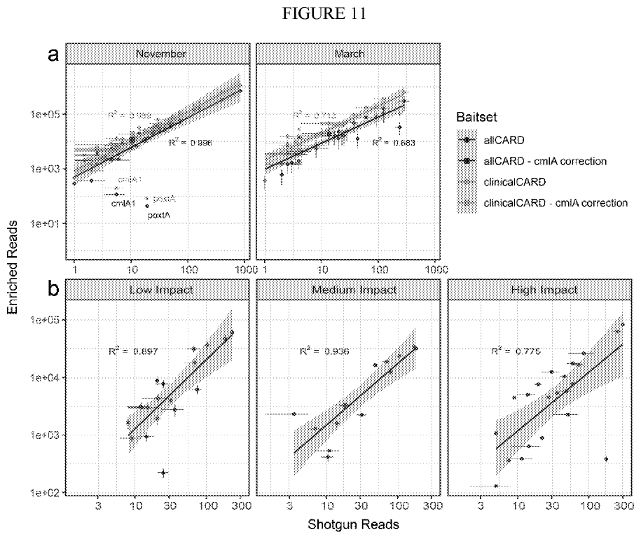

Finally, a commonly perceived limitation of enrichment is that hybridization can be sequence dependent, which may introduce biases in the final library, thereby eliminating the ability to perform relative quantification of ARGs in a sample. To investigate the relationship between ARG abundance in enriched vs unenriched data, the clinically relevant ARGs present with at least one read in both replicates of shotgun and enriched data at a subsampling depth of 3 M paired-end reads were analyzed (FIG. 11). In the wastewater samples, due to the high identity between reads, the R² value reached as high as 0.996 in the November sample with the allCARD probe set, while it was slightly lower after enrichment with clinicalCARD. In the March wastewater sample, R² values were considerably lower, most likely due to the inconsistent replicates. Enrichment was generally less efficient in the soil samples due to the lower identity between DNA and probes; however, the R² values remained high, reaching 0.897 in the low-impact sample, 0.936 in the medium-impact, and 0.775 in the high-impact sample from the hospital grounds.

In FIG. 11, ARGs were labeled on the plot if the enrichment factor was significantly lower (P<0.05) than the average enrichment factor within a sample and probe set. Two ARGs in the November wastewater sample were obvious outliers. The first, cmlA1, has several close homologs with >99% nucleotide identity to which thousands of reads were assigned. This occasional occurrence of reads mapping among highly similar alleles is known as the allele network problem [24, 30]. When mapping to highly redundant databases, even with new tools such as KMA as used by RGI bwt, reads can be misassigned to another closely related allele or ARG. To illustrate this, corrected points were superimposed onto the plot, i.e., showing where cmlA1 would reside on the plot if all the reads attributed to its variants were attributed to it instead. As expected, this correction brings it into far better agreement with the observed trend. The reads of the second outlier ARG, paxtA, appear to be from a distant homolog of the reference in CARD. Due to this sequence variance, it has a lower enrichment efficiency.

### Example 2: In-House Synthesis of a Mobilizable MGE RNA Bait Set (Reduced to Practice)

An RNA bait set configured to hybridize to target sequences associated with environmental mobilizable mobile genetic elements (“MGE probe set”) was synthesized using the in-house probe synthesis protocol described herein. In one embodiment, target sequences for probe design were assembled by compiling representative MGE-associated nucleotide sequences and genetic contexts from one or more public resources, including integron, insertion sequence, and transposon databases (e.g., INTEGRALL, ISFinder, and TnCentral). For example, candidate class 1 integron-associated contexts were identified by screening one or more of these resources using BLASTN with an integron integrase query sequence (e.g., intI1) and extracting matching sequences together with adjacent genetic context. Additional target sequences may include one or more ARG and/or gene cassette sequences observed in MGE contexts in metagenomic or genome datasets. Probe sequences were designed against the resulting target sequence set using a tiling and filtering workflow as described herein, optionally using a tiling step size of about 2 nucleotides and reducing redundancy to obtain a target probe count (e.g., about 24,000 probes).

Each probe sequence was converted into a synthetic oligonucleotide sequence by appending (i) an RNA polymerase promoter sequence (e.g., a T7 promoter) and (ii) an amplification primer binding sequence optionally comprising a type IIS restriction endonuclease recognition site adjacent to the probe sequence (e.g., an LguI site). The resulting sequences were obtained as an oligo pool, amplified by PCR to generate double-stranded DNA templates, digested with the restriction endonuclease to cleave at least a portion of the amplification primer binding sequence, and subjected to in vitro transcription using an RNA polymerase while incorporating a biotinylated nucleotide triphosphate (e.g., biotinylated UTP) to generate biotinylated RNA baits. Residual DNA template was digested (e.g., using DNase I), and the resulting biotinylated RNA baits were purified.

Quality control of the synthesized MGE bait set was performed using denaturing polyacrylamide gel electrophoresis (e.g., a 12.5% urea-PAGE gel stained with a nucleic acid stain). A successful reaction is indicated by a smeared band at an apparent molecular weight consistent with biotinylated RNA baits and an absence (or substantial reduction) of a band corresponding to the full-length DNA oligo pool (see, e.g., FIG. 5C).

### Example 3: In-House Synthesis of a Viral Bait Set (Reduced to Practice)

An RNA bait set configured to hybridize to viral target sequences (“viral probe set”) was synthesized using the in-house probe synthesis protocol described herein (see, e.g., “Probe synthesis” above). Briefly, a pooled collection of synthetic DNA oligonucleotides comprising a plurality of viral probe sequences flanked by (i) an RNA polymerase promoter sequence and (ii) an amplification primer binding sequence (optionally including a restriction endonuclease site proximal to each probe sequence) was obtained as an oligo pool. The oligo pool was amplified by PCR to generate double-stranded DNA templates, digested with a restriction endonuclease to cleave at least a portion of the flanking amplification primer binding sequence where present, and subjected to in vitro transcription using an RNA polymerase while incorporating a biotinylated nucleotide triphosphate (e.g., biotinylated UTP) to generate biotinylated RNA baits. Residual DNA template was digested (e.g., using DNase I), and the resulting biotinylated RNA baits were purified.

Quality control of the synthesized viral bait set was performed using denaturing polyacrylamide gel electrophoresis (e.g., a 12.5% urea-PAGE gel stained with a nucleic acid stain). A successful reaction is indicated by a smeared band at an apparent molecular weight consistent with biotinylated RNA baits and an absence (or substantial reduction) of a band corresponding to the full-length DNA oligo pool (see, e.g., FIG. 5B).

While the present disclosure has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS

1. Qian X, Gunturu S, Guo J, Chai B, Cole J R, Gu J, Tiedje J M. 2021. Metagenomic analysis reveals the shared and distinct features of the soil resistome across tundra, temperate prairie, and tropical ecosystems. Microbiome 9:108. https://doi.org/10.1186/s40168-021-01047-4
2. Liao H, Li H, Duan C S, Zhou X Y, An X L, Zhu Y G, Su J Q. 2022. Metagenomic and viromic analysis reveal the anthropogenic impacts on the plasmid and phage borne transferable resistome in soil. Environ Int 170:107595. https://doi.org/10.1016/j.envint.2022.107595
3. Hendriksen R S, Munk P, Njage P, van Bunnik B, McNally L, Lukjancenko O, Röder T, Nieuwenhuijse D, Pedersen S K, Kjeldgaard J, et al. 2019. Global monitoring of antimicrobial resistance based on metagenomics analyses of urban sewage. Nat Commun 10:1124. https://doi.org/10.1038/s41467-019-08853-3
4. Munk P, Brinch C, Møller F D, Petersen T N, Hendriksen R S, Seyfarth A M, Kjeldgaard J S, Svendsen C A, van Bunnik B, Berglund F, Global Sewage Surveillance Consortium, Larsson DGJ, Koopmans M, Woolhouse M, Aarestrup F M. 2022. Genomic analysis of sewage from 101 countries reveals global landscape of antimicrobial resistance. Nat Commun 13:7251. https://doi.org/10.1038/s41467-022-34312-7
5. Ras V, Carvajal-López P, Gopalasingam P, Matimba A, Chauke P A, Mulder N, Guerfali F, Del Angel V D, Reyes A, Oliveira G, De Las Rivas J, Cristancho M. 2021. Challenges and considerations for delivering bioinformatics training in LMICs: perspectives from Pan-African and Latin American bioinformatics networks. Front Educ 6:710971. https://doi.org/10.3389/feduc.2021.710971
6. Tewhey R, Nakano M, Wang X, Pabón-Peña C, Novak B, Giuffre A, Lin E, Happe S, Roberts D N, LeProust E M, Topol E J, Harismendy O, Frazer K A. 2009. Enrichment of sequencing targets from the human genome by solution hybridization. Genome Biol 10:1-13. https://doi.org/10.1186/gb-2009-10-10-r116
7. Gaudin M, Desnues C. 2018. Hybrid capture-based next generation sequencing and its application to human infectious diseases. Front Microbiol 9:2924. https://doi.org/10.3389/fmicb.2018.02924
8. Campana M G. 2018. BaitsTools: software for hybridization capture bait design. Mol Ecol Resour 18:356-361. https://doi.org/10.1111/1755-0998.12721
9. Yin X, Deng Y, Ma L, Wang Y, Chan L Y L, Zhang T. 2019. Exploration of the antibiotic resistome in a wastewater treatment plant by a nine-year longitudinal metagenomic study. Environ Int 133:105270.
10. Alcock B P, Raphenya A R, Lau TTY, Tsang K K, Bouchard M, Edalatmand A, Huynh W, Nguyen A-L V, Cheng A A, Liu S, et al. 2020. CARD 2020: antibiotic resistome surveillance with the Comprehensive Antibiotic Resistance Database. Nucleic Acids Res 48: D517-D525.
11. Salverda MLM, De Visser JAGM, Barlow M. 2010. Natural evolution of TEM-1 β-lactamase: experimental reconstruction and clinical relevance. FEMS Microbiol Rev 34:1015-1036.
12. Pawlowski A C, Stogios P J, Koteva K, Skarina T, Evdokimova E, Savchenko A, Wright G D. 2018. The evolution of substrate discrimination in macrolide antibiotic resistance enzymes. Nat Commun 9:112.
13. Daly M, Villa L, Pezzella C, Fanning S, Carattoli A. 2005. Comparison of multidrug resistance gene regions between two geographically unrelated Salmonella serotypes. J Antimicrob Chemother 55:558-561.
14. Scholz P, Haring V, Wittmann-Liebold B, Ashman K, Bagdasarian M, Scherzinger E. 1989. Complete nucleotide sequence and gene organization of the broad-host-range plasmid RSF1010. Gene 75:271-288.
15. De Oliveira DMP, Forde B M, Kidd T J, Harris PNA, Schembri M A, Beatson S A, Paterson D L, Walker M J. 2020. Antimicrobial resistance in ESKAPE pathogens. Clin Microbiol Rev 33: e00181-19.
16. Pendleton J N, Gorman S P, Gilmore B F. 2013. Clinical relevance of the ESKAPE pathogens. Expert Rev Anti Infect Ther 11:297-308.
17. Zhang Z, Zhang Q, Wang T, Xu N, Lu T, Hong W, Penuelas J, Gillings M, Wang M, Gao W, Qian H. 2022. Assessment of global health risk of antibiotic resistance genes. Nat Commun 13:1553.
18. Zhang A-N, Gaston J M, Dai C L, Zhao S, Poyet M, Groussin M, Yin X, Li L-G, van Loosdrecht M C M, Topp E, Gillings M R, Hanage W P, Tiedje J M, Moniz K, Alm E J, Zhang T. 2021. An omics-based framework for assessing the health risk of antimicrobial resistance genes. Nat Commun 12:4765.
19. Bar-On Y M, Phillips R, Milo R. 2018. The biomass distribution on Earth. Proc Natl Acad Sci USA 115:6506-6511.
20. Altschul S F, Gish W, Miller W, Myers E W, Lipman D J. 1990. Basic local alignment search tool. J Mol Biol 215:403-410.
21. Harris C R, Millman K J, van der Walt S J, Gommers R, Virtanen P, Cournapeau D, Wieser E, Taylor J, Berg S, Smith N J, et al. 2020. Array programming with NumPy. Nature 585:357-362.
22. Conrad T, Plumbom I, Alcobendas M, Vidal R, Sauer S. 2020. Maximizing transcription of nucleic acids with efficient T7 promoters. Commun Biol 3:439.
23. Wickham H. 2016. ggplot2: Elegant Graphics for Data Analysis. Springer-Verlag, New York.
24. Clausen P T L C, Aarestrup F M, Lund O. 2018. Rapid and precise alignment of raw reads against redundant databases with KMA. BMC Bioinformatics 19:307.
25. Tange O. 2011. GNU Parallel: The Command-Line Power Tool.; login: The USENIX Magazine 36:42-47.
26. Mckinney W. 2010. Data Structures for Statistical Computing in Python. Proceedings of the 9th Python in Science Conference, 51-56.
27. Cock P J A, Antao T, Chang J T, Chapman B A, Cox C J, Dalke A, Friedberg I, Hamelryck T, Kauff F, Wilczynski B, de Hoon M J L. 2009. Biopython: freely available Python tools for computational molecular biology and bioinformatics. Bioinformatics 25:1422-1423.
28. Pedregosa F, Varoquaux G, Gramfort A, Michel V, Thirion B, Grisel O, Blondel M, Prettenhofer P, Weiss R, Dubourg V. 2011. Scikit-learn: Machine Learning in Python. J Mach Learn Res 12:2825-2830.
29. Jia B, Raphenya A R, Alcock B, Waglechner N, Guo P, Tsang K K, Lago B A, Dave B M, Pereira S, Sharma A N, et al. 2017. CARD 2017: expansion and model-centric curation of the Comprehensive Antibiotic Resistance Database. Nucleic Acids Res 45: D566-D573.
30. Lanza V F, Baquero F, Martínez J L, Ramos-Ruíz R, González-Zorn B, Andremont A, Sánchez-Valenzuela A, Ehrlich S D, Kennedy S, Ruppé E, van Schaik W, Willems R J, de la Cruz F, Coque T M. 2018. In-depth resistome analysis by targeted metagenomics. Microbiome 6:11.

1. A method of providing a probe set comprising a plurality of probes for hybridization-based targeted enrichment of target nucleic acid, the method comprising:
a) selecting a target nucleic acid;
b) generating candidate probe sequences to the target nucleic acid by tiling candidate probes across at least a portion of the target nucleic acid;
c) filtering the candidate probe sequences based on one or more sequence or thermodynamic constraints;
d) filtering the candidate probe sequences based on sequence similarity to one or more off-target nucleic acid sequence datasets;
e) reducing redundancy among remaining candidate probe sequences to obtain the probe set; and
f) optionally, synthesizing one or more of the probes from the probe set.

2. The method of claim 1, wherein each probe sequence is >40 nucleotides in length.

3. The method of claim 1, wherein the filtering of step c) comprises excluding candidate probe sequences with:
i) a melting temperature below a threshold melting temperature;
ii) ambiguous bases; and/or
iii) a type IIS restriction endonuclease recognition site.

4. The method of claim 1, wherein the filtering of step d) comprises comparing candidate probe sequences to at least one off-target dataset and excluding candidate probe sequences having a sequence similarity to one or more sequences in the dataset that is above at least a sequence identity of 50 nucleotides over a probe length of at least 50 nucleotides.

5. The method of claim 1, wherein reducing redundancy comprises iteratively collapsing clusters of candidate probes to a single representative candidate probe sequence, wherein each cluster exhibits less sequence similarity than the previous cluster, until a target probe count is reached.

6. The method of claim 1, wherein the target nucleic acid is selected from the group consisting of genes, genomic regions, DNA sequences and RNA sequences.

7. The method of claim 6, wherein the genes comprise antimicrobial resistance genes.

8. The method of claim 6, wherein the genomic regions comprise regions associated with mobile genetic elements.

9. The method of claim 1, wherein the candidate probes are tiled across the target nucleic acid sequence at a step size of 1-10 nucleotides.

10. A method of generating labelled single-stranded RNA baits for hybridization-based targeted enrichment of target nucleic acid, the method comprising:
a) providing a plurality of probes each configured to hybridize to at least a portion of the target nucleic acid, appending an RNA polymerase promoter at the 5′ end of each probe and an amplification primer to the 3′ end of each probe to yield a pool of single-stranded DNA oligonucleotides;
b) amplifying the pool of single-stranded DNA oligonucleotides using (i) a first primer complementary to at least a portion of the RNA polymerase promoter and (ii) a second primer complementary to at least a portion of the amplification primer to generate double-stranded DNA templates; and
c) performing in vitro transcription of the double-stranded DNA templates in the presence of an RNA polymerase that recognizes the RNA polymerase promoter and mixture of four nucleotide triphosphates comprising adenine, guanine, cytosine and uracil bases, wherein at least one of the four nucleotide triphosphates is labelled, to generate labelled single-stranded RNA baits which are immobilizable.

11. The method of claim 10, wherein at least a portion of the amplification primer is cleaved from the double-stranded DNA templates prior to transcription step c).

12. The method of claim 10, wherein the RNA polymerase promoter is a T7 promoter.

13. The method of claim 10, wherein the RNA polymerase promoter comprises one or more guanine bases directly downstream of a transcription initiation site.

14. The method of claim 10, wherein the labelled nucleotide triphosphate is biotinylated.

15. The method of claim 14, wherein the biotinylated nucleotide triphosphate is biotinylated UTP.

16. The method of claim 10, wherein the amplification primer is cleaved by a restriction endonuclease that recognizes a restriction endonuclease recognition site within the amplification primer.

17. The method of claim 16, wherein the restriction endonuclease is a type IIS restriction endonuclease.

18. The method of claim 17, wherein the type IIS restriction endonuclease is LguI.

19. A kit for generating biotinylated single-stranded RNA baits for hybridization-based targeted enrichment of target DNA, comprising a pool of single-stranded DNA oligonucleotides, wherein each oligonucleotide comprises a probe sequence having an RNA polymerase promoter appended to the 5′ end of the probe and an amplification primer appended to the 3′ end of the probe, wherein the kit optionally comprises one or more of:
a) a first primer complementary to at least a portion of the RNA polymerase promoter
b) a second primer complementary to at least a portion of the amplification primer;
c) a DNA polymerase suitable for PCR;
d) an RNA polymerase that recognizes the RNA polymerase promoter;
e) mixture of four nucleotide triphosphates comprising adenine, guanine, cytosine and uracil bases, wherein at least one of the four nucleotide triphosphates in the mixture is biotinylated;
f) a restriction endonuclease; and/or
g) a DNase.
